(19) United States
(12) Patent Application Publication
(10) Pub. No.: US 2026/0055076 A1
(43) Pub. Date: Feb. 26, 2026

(54) PROCESS AND SYSTEM FOR REMOVING LACTIDE FROM A LACTIDE-CONTAINING GAS STREAM

(71) Applicant: PURAC Biochem B.V., Gorinchem (NL)

(72) Inventors: Johannes Jeichienus DE VRIES, Gorinchem (NL); Carlos Alberto GONZALEZ RUGERIO, Gorinchem (NL); Gerrit GOBIUS DU SART, Herwijnen (NL); Denis LOOZEN, Uccle (BE)

(21) Appl. No.: 19/379,071

(22) Filed: Nov. 4, 2025

Related U.S. Application Data
(63) Continuation of application No. PCT/EP2024/062122, filed on May 2, 2024.

(30) Foreign Application Priority Data
May 5, 2023 (EP) 23171823.0

(51) Int. Cl. C07D 319/12; B01D 5/00; C08G 63/08; C08G 63/78

(52) U.S. Cl. CPC C07D 319/12; B01D 5/0057; C08G 63/08; C08G 63/78

(57) ABSTRACT

The invention pertains to a process for removing lactide from a vapour stream comprising lactide and water, comprising the steps of ⋅subjecting a vapour stream comprising lactide and water to a condensation-reaction step in a condensation-reaction apparatus, wherein the vapour stream comprising lactide and water is contacted with a reaction liquid comprising lactic acid, the condensation reaction step being carried out under such conditions that at least 80% of the lactide present in the vapour stream provided to the condensation-reaction step is absorbed in the reaction liquid and at most 40% of the water present in the vapour stream provided to the condensation-reaction step is absorbed in the reaction liquid, wherein the residence time in the condensation step is at least 2 hours, the residence time being defined as the total mass of reaction liquid divided by the mass of the product condensing from the vapor stream containing lactide and water per hour, ⋅withdrawing a liquid effluent from the condensation-reaction step, which liquid effluent comprises less than 15 wt. % lactide and less than 30 wt. % of water, the effluent having a total acid content of at least 72 wt. %. ⋅withdrawing a steam effluent from the condensation-reaction step, and providing the steam effluent to a further condensation reaction in a further condensation apparatus to form a condensate comprising water. The present invention provides a robust and efficient process and system for removing lactide from a lactide-containing gas stream and recovering said lactide in a processable form. The process of the invention is able to handle feedstock with varying compositions and has a limited demand on energy and apparatus.

### FIELD OF THE INVENTION

The present invention relates to a process and system for removing lactide from a lactide-containing gas stream. Lactide containing gas streams can have many origins. They can derive, e.g., from the manufacture of polylactic acid (PLA) from lactide monomers, where the final polymer still comprises unreacted lactide which is to be removed. Lactide-containing gas streams may also be derived from a process for manufacturing lactide. In such a process, waste streams may be generated which still contain lactide in an amount sufficiently large for it to be recovered.

### BACKGROUND OF THE INVENTION

The demand for biodegradable polymers with excellent material properties is rapidly growing. Biodegradable polymers can be used in various applications, from biomedicine, additive technologies, film, fibers, packaging, automotive to agriculture, etc. Polylactide, which is also referred to as polylactic acid and abbreviated as PLA, has been receiving increasing attention in recent years for use in these applications, because of its excellent performances in renewability, mechanical properties, biocompatibility and biodegradability. PLA is an aliphatic polyester, which can be manufactured from renewable resources. Such manufacture may involve the fermentation of starch, sugar or other renewable organic substrates into lactic acid. Polylactide can be produced by direct polycondensation of lactic acid, i.e. lactate monomers. However, this has the drawback that a high molecular weight is not easily reached. Therefore, PLA is usually prepared by ring-opening polymerization (ROP) of lactide, the cyclic dimer of lactic acid, which in turn is usually manufactured by polycondensation of lactic acid to form lactic acid oligomers, followed by de-polymerization of these oligomers by a so-called ‘backbiting’ mechanism in the presence of a suitable catalyst. After purification, the produced lactide can be converted into PLA of controlled molecular weight by means of a ring-opening polymerization reaction (ROP) in the presence of a polymerization catalyst and initiator. Ring-opening polymerization allows to control the polymerization process and thereby the structure of the produced PLA. This method can be used to manufacture PLA of high molecular weight. The molecular weights of the polymer fabricated by the ring opening polymerization can be controlled by residence time, catalyst and initiator concentration, and temperature. The sequence and ratio of L- and D-lactic acid units in the final polymer can also be controlled.

During polymerisation of PLA (polylactide), the polymer, because of the chemical ring-chain equilibrium, always comprises lactide in a concentration which is dependent upon the polymerisation temperature and is between approx. 1 and 5%. This value is independent of whether the PLA is produced by ring-opening polymerisation from lactide or by direct polycondensation from lactic acid. During ring-opening polymerisation, the concentration of the lactide can also assume higher values if the reaction is interrupted even before reaching the chemical equilibrium, e.g. by addition of a substance which deactivates the polymerisation catalyst.

The unreacted lactide has to be removed from the PLA polymer after the polymerization in order to obtain a PLA product of marketable quality. Lactide concentrations in the PLA of more than 0.5% by weight make the polymer unusable for commercial purposes. During processing of PLA in the melt, such as spinning of threads, pouring of films, injection moulding etc, they lead to smoke which causes coughing, pollutes and corrodes devices. Lactide-containing PLA granulate absorbs moisture when stored in ambient air, lactide being hydrolysed to form the linear dimer of lactic acid. During processing from the melt, this hydrolysis product leads to the rapid decomposition of the PLA chains because of the high melting temperature required for this purpose of more than 170° C. (melting point of PLA), so that the polymer loses technically important properties, such as strength, transparency etc., and becomes unusable. The lower the residual concentration of lactide in the PLA, the more durable are the products produced therefrom and the better it behaves during processing. The required removal of lactide generally occurs by subjecting a PLA melt containing lactide to a so-called devolatilization step, where lactide is evaporated from the PLA melt at elevated temperature and reduced pressure. The devolatilization step generates a PLA stream with a reduced lactide content, and a lactide-containing gas stream. It is necessary to remove the lactide from such lactide-containing gas streams, both for environmental and for economic reasons. In particular, it is necessary to recover lactide from a lactide-containing gas stream in an economic manner and reliable manner, whether in the form of lactide itself, or in the form of lactic acid or related compounds.

As indicated above, lactide, the cyclic dimer of lactic acid, is generally manufactured by polycondensation of lactic acid to form lactic acid oligomers, followed by de-polymerization of these oligomers to form lactide. Lactide manufacturing processes are generally characterised by a substantial number of separation and purification steps. This is because the lactide that is to be produced should be relatively pure. Contaminants that have to be removed from lactide in a lactide manufacturing process include, in particular, lactic acid, the presence of which detrimentally affects the stability of PLA to be produced from the lactide. Contaminants may further include various compounds originating from the lactic acid feed, in particular where the lactic acid feed originates from a fermentation process. Another reason lactide manufacturing processes generally contain a substantial number of separation and purification steps is the desire for stereochemical purity. There are three stereoisomers of lactide, namely L-lactide, (the cyclic dimer of two (S)-lactic acid molecules), D-lactide (the cyclic dimer of two (R)-lactic acid molecules), and meso-lactide (the cyclic dimer of an (S)-lactic acid molecule and an (R)-lactic acid molecule). The ratio in which the various lactides are provided to a polymerisation process influences the physical properties of the polylactide. For example. a polylactide comprising mainly (S)-lactic units and, say, 10% of (R)-lactic acid units will, for example, be less crystalline than a polylactide comprising only (S)-lactic acid units. Thus, lactide manufacturing processes generally contain separation steps in which various stereoisomers of lactide are separated from each other. Accordingly, in a lactide manufacturing process lactide-containing gas streams are produced at a number of locations. Again, it is necessary to remove the lactide from such lactide-containing gas streams, both for environmental and for economic reasons.

There are challenges associated with the removal of lactide from lactide-containing gas streams. Both in lactide synthesis and in lactide separation in PLA manufacture the lactide-containing gas streams are at a low pressure. The pressure in a lactide synthesis reactor generally is in the range of 3-50 mbar; in the associated distillative purification it generally is in the range of 6-80 mbar. In PLA manufacture lactide is generally removed from the PLA melt at a pressure in the range of 1-20 mbar. One risk in the processing of lactide-containing gas streams is blockage of the vacuum system by the deposition of lactide, especially in condensers and vacuum pumps. This risk is heightened by the relatively high melting point of L-lactide and D-lactide (96-97° C.), and by the fact that the solubility of lactide in water, especially cold water (5-30° C.) is limited. Accordingly, removing lactide from gas streams which are at low pressure and at relatively low temperatures, in particular in the presence of water which may originate from a steam ejector, should be handled with care.

Various methods for recovering lactide from lactide-containing gas streams have been described in literature.

US20140012043 describes a method for removing a cyclic ester of a 2-hydroxy alkanoic acid such as lactide from a vapor by contacting the vapour with an alkaline solution, preferably having a pH above 10. A disadvantage to this process is that the use of an alkaline solution brings new compounds into the system, which have to be processed

US20100252076 is directed to a condensation and washing device in which vapours derived from PLA production can be processed. Process vapours are condensed, and washed with a lactic-acid containing washing liquid. It is stated that water and lactic acid are added to the process continuously to avoid the solubility limit of the lactide. The process as described in this reference requires stringent process control to address the solubility issues raised. Stringent process control leads to a relatively inflexible process, and as the composition of feed gas may vary, this is undesirable.

US20150151247 is directed to a method for removing an ester from a vapour mixture by, in a dissolution step, bringing the vapour into contact with an aqueous solution containing the corresponding acid to dissolve a portion of the ester into the solution. To avoid build-up of lactide in the solution, the lactide is hydrolyzed. To do this, the aqueous solution is subjected to a series of steps including a heating step, a reaction (hydrolyzation) step to react the lactide, and a cooling step. The performance of the step sequence makes the process quite complex and highly energy-intensive. Further, the variable equipment cost is high since the solubility of lactide in water is very low at the temperature applied. This means that there is a risk of forming solid lactide in the dissolution step, which may block the system. This can be addressed by increasing the circulation flow, but this will substantially increase the heating and cooling costs. This makes the process quite sensitive to process upset if a larger than normal amount of lactide is provided to the process.

It is an object of the present invention to provide a robust and efficient process and system for removing lactide from a lactide-containing gas stream and recovering said lactide in a processable form. A robust process is a process which is able to handle feedstock with varying compositions. An efficient process is a process which has a limited demand on energy and apparatus. The present invention provides such a process.

### SUMMARY OF THE INVENTION

The present invention is directed to a process for removing lactide from a vapour stream comprising lactide and water, comprising the steps of
subjecting a vapour stream comprising lactide and water to a condensation-reaction step in a condensation-reaction apparatus, wherein the vapour stream comprising lactide and water is contacted with a reaction liquid comprising lactic acid, the condensation reaction step being carried out under such conditions that at least 80% of the lactide present in the vapour stream provided to the condensation-reaction step is absorbed in the reaction liquid and at most 40% of the water present in the vapour stream provided to the condensation-reaction step is absorbed in the reaction liquid, wherein the residence time in the condensation step is at least 2 hours, the residence time being defined as the total mass of reaction liquid divided by the mass of the product condensing from the vapor stream containing lactide and water per hour,
withdrawing a liquid effluent from the condensation-reaction step, which liquid effluent comprises less than 15 wt. % lactide and less than 30 wt. % of water, the effluent having a total acid content of at least 72 wt. %.
withdrawing a steam effluent from the condensation-reaction step, and providing the steam effluent to a further condensation reaction in a further condensation apparatus to form a condensate comprising water.

It has been found that the process according to the invention can handle vapour streams with varying compositions without undesirable side effects. The process can be operated in relatively simple and energy-efficient manner, and can easily be integrated into existing processes which generate lactide-containing vapor streams. The effluent streams from the process according to the invention are relatively pure, and can be tailored for easy further processing. The present process uses a limited number of apparatuses and equipment, and has a large operating window. This reduces complexity of the process and allows the overall process to provide high operational reliability and flexibility. It avoids the use of complex equipment, such as e.g. apparatuses that use heating/cooling cycles, can be operated in such a manner that the formation of solidified lactide at undesired locations can be prevented.

Further advantages of the present invention and specific embodiments thereof will become evident from the further specification.

The invention also pertains to a system suitable for use in the present invention, and to its integration into various processes which generate lactide-containing gas streams.

### DESCRIPTION OF THE FIGURES

FIG. 1 illustrates a first embodiment of the present invention.

FIG. 2 illustrates a variation on FIG. 1, in which the condensation-reaction apparatus comprises two sections.

FIG. 3 illustrates a specific embodiment of a unit suitable for performing a condensation-reaction step in the process of the invention.

FIG. 4 illustrates a further embodiment of present invention, in which the condensation-reaction step is preceded by a lactide condensation step in which the vacuum is provided through a steam ejector.

FIG. 5 illustrates an embodiment of the present invention in which the process according to the invention is integrated into a PLA manufacturing process.

FIG. 6 illustrates an embodiment of the present invention in which the process according to the invention is integrated into a lactide manufacturing process.

### DETAILED DESCRIPTION OF THE INVENTION

When describing the invention, the terms used are to be construed in accordance with the following definitions, unless a context dictates otherwise.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Reference throughout this specification to “one embodiment” or “an embodiment” means that a particular feature, structure, or characteristic described in connection with the embodiment is included in at least one embodiment of the present invention. Thus, appearances of the phrases “in one embodiment” or “in an embodiment” in various places throughout this specification are not necessarily all referring to the same embodiment, but may.

Furthermore, the particular features, structures or characteristics may be combined in any suitable manner, as would be apparent to a person skilled in the art from this disclosure, in one or more embodiments. Furthermore, while certain embodiments described herein include some but not other features included in other embodiments, combinations of features of different embodiments are meant to be within the scope of the invention, and form different embodiments, as would be understood by those in the art.

The terms “comprising”, “comprises” and “comprised of” as used herein are synonymous with “including”, “includes” or “containing”, “contains”, and are inclusive or open-ended and do not exclude additional, non-recited members, elements, or method steps. It will be appreciated that the terms “comprising”, “comprises” and “comprised of” as used herein comprise the terms “consisting essentially of”, “consisting of”, “consists” and “consists of”.

As used in the specification and the appended statements, the singular forms “a”, “an,” and “the” include plural referents unless the context clearly dictates otherwise. By way of example, “a step”means one step or more than one step.

The terms “apparatus”, “device”may be used herein as synonyms.

In general, the present invention is directed to a process in which various steps are carried out in a certain manner. The invention is not limited to a specific apparatus and the wording in the present specification should be interpreted in functional terms. For example, the word condenser refers to any apparatus of device which can effect condensation as described for the respective step in the process. Common for any condenser is to take heat out of the system and due to that to convert part of the vapor to liquid. The heat can be taken out by contacting the vapor by a cold surface. Suitable examples include shell & tubes heat exchangers and plate heat exchangers. An other way to take heat out of the system is to insert a cold liquid and condensing vapors by that. In this case the liquid needs do be discharged from the system after the condensing step. When circulation is applied for this cooling liquid, there is often an external cooler in the circulation system. packed columns and condensation columns are an example of this.

Unless specified otherwise, or unless it is clear to the skilled person that it is otherwise, a step can be carried out in single unit or in multiple units. Conversely, it may be possible to carry out more than one step in a single unit.

Unless defined otherwise, all technical and scientific terms used herein have the same meaning as is commonly understood by one of skill in the art.

The recitation of numerical ranges by endpoints includes all integer numbers and, where appropriate, fractions subsumed within that range (e.g. 1 to 5 can include 1, 2, 3, 4 when referring to, for example, a number of elements, and can also include 1.5, 2, 2.75 and 3.80, when referring to, for example, measurements). The recitation of endpoints also includes the end point values themselves (e.g. from 1.0 to 5.0 includes both 1.0 and 5.0). Any numerical range recited herein is intended to include all sub-ranges subsumed therein.

The terms “wt %,” “vol %”, or “mol %” refers to a weight percentage of a component, a volume percentage of a component, or molar percentage of a component, respectively, based on the total weight, the total volume of material, or total moles, which includes the component.

The present specification contains headings. These are present only to improve readability of the document. They do not in any way limit how the specification is to be interpreted, or whether or not various elements of the specification can be combined.

Pressure values in mbar refer to absolute pressure, often indicated as mbar(a) or mbara. In the present specification mbar is used.

In the present specification a stream to a specific step may sometimes be indicated as a feed. The terms are to be regarded as synonymous unless it is clear from the context that they are not.

The terms gas and vapour are used interchangeably herein. The terms are to be regarded as synonymous unless it is clear from the context that they are not.

Reference to lactide encompasses L-lactide, D-lactide, meso-lactide or any mixture or combination thereof, unless it is clear from the context that a specific lactide species is intended.

The direction of flow through a step or apparatus is not critical to the process according to the invention. It will be clear to the skilled person that all steps and units may be operated in any flow direction, unless it is evident to the skilled person that only one operating mode is possible, or unless the flow direction is indicated specifically.

Unless specified otherwise, where a stream flows from one step or unit to another step or unit, the stream provided to the second step or unit may be the entirety of the stream withdrawn from the first step or unit, or it may be only part of said stream.

### Condensation-Reaction Step

The first step in the process according to the invention is to subject a vapour stream containing lactide and water to a condensation-reaction step. In the reaction-condensation step, the vapour stream comprising lactide and water is contacted with a reaction liquid comprising lactic acid. The reaction condensation step is carried out under such conditions that at least 80% of the lactide present in the vapour stream provided to the condensation-reaction step is absorbed in the reaction liquid. The step is carried out in such a way that the residence time in the condensation-reaction step is at least 2 hours, the residence time being defined as the total mass of reaction liquid divided by the mass of the product condensing from the vapor stream containing lactide and water per hour. By providing a minimum residence time, it can be ensured that the lactide condensed in the reaction liquid reacts to form lactic acid or linear lactic acid oligomers. This means that the increase of lactide concentration in the liquid is limited and the flowrate can be selected such that maximum solubility of lactide in the reaction this liquid is not exceeded. It is within the scope of the skilled person to design the process in such a manner that the requirements for the residence time are met. In one embodiment, there is a hold-up which ensures that there is a certain residence time in the condensation step, which results in the conversion of lactide absorbed into the reaction liquid into lactic acid or lactic acid oligomers. In one embodiment, the reaction liquid will be circulated over a condensing area, a hold up area, generally in the bottom, and a cooler. Any other way of having mixed cooled liquid flowing through the contact area of vapor and liquid has the same functionality. As explained above, by applying a certain residence time, the amount of lactide that can be absorbed into the reaction liquid is not limited by the solubility of the lactide in the reaction liquid. This makes for a flexible process which can be adapted to the amount of lactide in the feed by adapting the flow rate ratio. This prevents the occurrence the formation of solid lactide at undesired locations.

The condensation-reaction step is carried out under such conditions that at most 40% of the water present in the vapour stream provided to the condensation-reaction step is absorbed in the reaction liquid. This is advantageous because it makes for the formation of a highly concentrated liquid effluent in the condensation-reaction step in combination with a water effluent that can be processed separately. The liquid effluent withdrawn from the condensation-reaction step comprises less than 15 wt. % lactide and less than 30 wt. % of water, the effluent having a total acid content of at least 72 wt. %.

The vapour feed to the condensation-reaction step comprises water and lactide. The feed will generally also contain non-condensables, such as nitrogen, oxygen, carbon dioxide, and in some cases carbon monoxide. The feed may contain further components, such as lactic acid. Whether or not such further components will be present, will depend on the source of the vapour feed. In general, the vapour feed will contain 10 to 90 wt. % of water, calculated on the total of lactide, water, and lactic acid in the feed, e.g., 30 to 80 wt. %. The exact value will depend on the origin of the vapour feed. In general, the vapour feed will contain 2 to 50 wt. % of lactide, calculated on the total of lactide, water, and lactic acid in the stream, e.g., 5 to 30 wt. %. Again, the exact value will depend on the origin of the vapour feed. The vapour feed generally contains 0-20 wt. % of lactic acid, calculated on the total of lactide, water, and lactic acid in the vapour feed. In one embodiment, in particular where lactide streams derived from PLA are processed, the amount of lactic acid in the vapour feed will be low, e.g. 0-10 wt. % of lactic acid, calculated on the total of lactide, water, and lactic acid in the feed. in particular 0-5 wt. %, more in particular 0 to 2 wt. %. In other embodiments the lactic acid content may be higher, e.g., 2-15 wt. %, or 2-5 wt. %.

The feed may contain minor amounts of other organic contaminants, e.g., up to 5 wt. %, calculated on the total of lactide, water, and lactic acid in the feed, in general up to 2 wt. %.

The vapour stream containing lactide and water is contacted with a reaction liquid which contains lactic acid. In general, the reaction liquid contains at least 5 wt. % of lactic acid, in particular at least 10 wt. %, more in particular at least 20 wt. % of lactic acid, depending on the further composition. The composition of the reaction liquid may vary over time, depending on precise process configuration. For example, it is possible for the reaction liquid to have a relatively low lactic acid concentration at the start of the process, with the concentration increasing due to recycle of the reaction liquid.

In one embodiment, the composition of the reaction liquid meets the same requirements as the composition of the liquid effluent from the condensation-reaction step. The ranges and preferences discussed for that composition also apply here.

The reaction liquid may be fresh, in that it is provided from outside the process, but it may also consist at least in part from a recycle stream, in particular an effluent stream which is withdrawn from the condensation-reaction step. In one embodiment, at least 50 vol. % of the reaction liquid provided to the condensation-reaction step consists of liquid effluent from the condensation-reaction step, in particular at least 80 vol. %, more in particular at least 90 vol. %.

The process is carried out under such conditions that at least 80% of the lactide present in the vapour stream provided to the condensation-reaction step is absorbed in the reaction liquid. The aim of the condensation-reaction step is to recover lactide. It is therefore preferred that at least 90% of the lactide present in the vapour stream provided to the condensation-reaction step is absorbed in the reaction liquid, in particular at least 95%, more in particular at least 98%.

If the vapour stream comprises further components, such as lactic acid or other organic components, these will generally also condense in the condensation-reaction step to a larger or smaller extent. Given the limited content of these components, they require no further discussion.

The process is carried out under such conditions that at most 40% of the water present in the vapour stream provided to the condensation-reaction step is absorbed in the reaction liquid. It is a feature of the present invention that the liquid effluent from the condensation-reaction step is relatively concentrated. Accordingly, it may be preferred to carry out the process under such conditions that at most 30% of the water present in the vapour stream provided to the condensation-reaction step is absorbed in the reaction liquid, in particular at most 25%, more in particular at most 20%, more in particular at most 15%. The amount of water that is condensed in the condensation-reaction step may be much lower, e.g., at most 10%, in particular at most 5%, or even at most 2%, calculated on the amount of water provided in the feed.

In some embodiments, in particular where the reaction liquid is highly concentrated, it may be preferred to condense some water in the condensation-reaction step, to manage the solubility of the lactide. Therefore, in some embodiments, the amount of water that is condensed in the condensation-reaction step is in the range of 0.5-10 wt. %, in particular in the range of 1-5 wt. %. This is of particular relevancy when the total acid content in the reaction liquid is at least 80 wt. %, in particular at least 90 wt. %, in particular at least 92 wt. %, more in particular at least 94 wt. %, still more in particular at least 96 wt. %, e.g., where a substantial part of the reaction liquid is recycled.

In general, it is well within the scope of the skilled person to select the reaction conditions during the condensation-reaction step to ensure that the percentages of lactide and water that are to be condensed are met. As the skilled person will appreciate, lower temperatures, and to a somewhat lesser extent higher pressures will increase the percentage of lactide that will condense. On the other hand, the same conditions will also contribute to the condensation of water, with pressure playing a larger role. Therefore, as will be evident to the skilled person, to achieve the desired condensation of lactide and water a temperature and pressure should be selected at which the desired lactide condensation is obtained, but which are not so severe that more water is condensed than is aimed for. This can be obtained at various temperature/pressure combinations.

The temperature in the condensation-reaction step generally is between 20 and 150° C., such as between 30 and 100° C., or between 40 and 80° C. The pressure in the condensation-reaction step generally is in the range of 5 to 100 mbar, such as from 10 to 50 mbar, or from 15 to 40 mbar. In an example a second condensation apparatus is operated in a process of the invention at a temperature of 150° C. and pressure of 100 mbar. In another example, a second condensation apparatus is operated in a process of the invention at a temperature of 100° C. and pressure of 50 mbar. In another example, a second condensation apparatus is operated in a process of the invention at a temperature of 60° C. and pressure of 30 mbar. In another example, a second condensation apparatus is operated in a process of the invention at a temperature of 40° C. and pressure of 20 mbar. In another example, a second condensation apparatus is operated in a process of the invention at a temperature of 20° C. and pressure of 15 mbar. In one embodiment, the condensation-reaction step is carried out at a temperature in the range of 30-70° C. at a pressure in the range of 20-40 mbar. In another embodiment, the condensation-reaction step is carried out at a temperature in the range of 40-80° C. at a pressure in the range of 10-20 mbar.

In the condensation-reaction step, lactide will be adsorbed into the reaction liquid and reacted to form lactic acid and linear lactic acid oligomers. In the context of the present specification the word absorbed is intended to refer to any process which results in the component at issue entering into and being retained in the reaction liquid in some form. It thus encompasses, but is not limited to dissolution.

The reaction encompasses a hydrolysis reaction to break open the cyclic dimer to form a linear dimer. It may encompass further hydrolysis reactions to form lactic acid, or condensation reactions to form lactic acid oligomers. That this reaction step takes place is a feature of the present invention. Because the lactide is reacted, the solubility limit for lactide in the reaction liquid will not be reached. This decreases the risk of lactide precipitation. In one embodiment, at least 50% of the lactide provided to the condensation-reaction step is converted into lactic acid or lactic acid oligomers (calculated from the amount of lactide provided to the condensation-reaction step and the lactide content of the lactic acid-containing liquid withdrawn from the condensation-reaction step). It may be preferred for at least 70% of the lactide provided to the condensation-reaction step to be converted into lactic acid or lactic acid oligomers, in some embodiments at least 80%, or at least 90%.

It is a feature of the present invention that measures are taken to ensure that there is sufficient time for the lactide to react. This can be controlled through the residence time. In the process according to the invention, the residence time in the condensation-reaction step is at least 2 hours, the residence time being defined as the total mass of reaction liquid divided by the mass of the product condensing from the vapor stream containing lactide and water per hour.

The residence time in the condensation-reaction step may be may be at least 5 hours, more in particular at least 10 hours, still more in particular at least 20 hours, or at least 30 hours.

As an operating maximum a value of at most 1000 hours may be mentioned. A maximum of at most 200 hours, in particular at most 150 hours may be mentioned.

In one embodiment, the process according to the invention may be carried out at a specific ratio between the condensing liquid and the total reaction liquid. This ratio may be defined as follows: ratio (no units)=total reaction liquid flow rate (reaction liquid in circulation in the system+washing liquid added to the system)/flow rate of the liquid condensing from the vapour stream comprising water and lactide. In one embodiment, this ratio is at least 5:1. The ratio may be much higher, e.g., at least 10:1, in particular at least 20:1, more in particular at least 30:1. A higher ratio may be desirable because it may reduce the risk of lactide precipitation. As an operating maximum a ratio of at most 1000:1 may be mentioned. It may be preferred for the ratio to be at most 200:1, in particular at most 150:1, more in particular at most 100:1.

The condensation-reaction step may be carried out in a single step in a single unit, but it may also be carried out in a number of steps in a number of units, wherein the units are the same of different. It is also possible for the condensation-reaction step to be carried out in a single unit having a number of sections.

In one embodiment, the condensation-reaction step is carried out in a condenser followed by a holding vessel, with a lactic acid containing reaction liquid being provided to the condenser. In this configuration, lactide will be absorbed into the lactic acid containing reaction liquid in the condenser, while the reaction of lactide to form lactic acid and linear lactic acid oligomers takes place in the holding vessel. The values given above for temperature, pressure, residence time, etc., apply to the combination of both units. The lactic acid containing reaction liquid may be provided from outside the unit, but it is also possible for the reaction liquid provided to the condenser to be, at least in part, a recycle stream derived from the holding vessel. In fact, it is preferred for at least 80% of the volume of the reaction liquid provided to the condenser to be derived from the holding vessel, in particular at least 90 vol. %. If the process is carried out in a continuous manner, it may be preferred if—after startup—the reaction liquid being provided to the condenser consists in its entirety of recycle liquid from the holding unit.

In one embodiment, the reaction liquid in the system consists of a recycle stream and an additional stream provided to the system. In the context of the present specification, the term additional stream is used in contrast with a recycle stream. An additional stream may, e.g., be a stream of washing liquid. In one embodiment, the flowrate (weight/h) of the recycle stream is at least 50% of the flowrate (weight/h) of the total of recycle stream and additional stream, in particular at least 70%, in some embodiments at least 80%.

In one embodiment, the condensation-reaction step is carried out in a condensation-reaction unit comprising, from top to bottom, a top condenser, a lower condenser, and a sump. In this embodiment, the feed vapour stream comprising lactide and water enters the unit above the sump at the lower end of the lower condenser, and flows upwards. Lactic acid containing reaction liquid is provided at the top of the lower condenser and at the top of the top condenser. The lactic-acid containing reaction liquid absorbs lactide from the vapour stream, and flows down to the sump. In the sump, the lactide absorbed from the vapour stream is allowed to react to form lactic acid and lactic acid oligomers.

The temperatures in the top condenser and in the lower condenser may be the same or different. In one embodiment, the temperature in the top condenser is lower than the temperature in the lower condenser. E.g., in one embodiment, the temperature in the lower condenser may be at least 5° C. below the temperature in the top condenser, in particular at least 10° C., in some embodiments at least 15° C. In general, the temperature difference will be at most 50° C., in particular at most 45° C., more in particular at most 40° C. In one embodiment, the temperature in the lower condenser is in the range of 60-100° C., in particular 70-90° C., while the temperature in the top condenser is in the range of 20-70° C., in particular 30-60° C.

The lactic acid containing reaction liquid provided to the two condensers may be the same or different. In one embodiment, the lactide concentration of the reaction liquid provided to the top condenser is lower than the lactide concentration of the reaction liquid provided to the top of the lower condenser. One way to effect this is for the reaction liquid provided to the lower condenser to be at least partly, and preferably in its entirety, derived from the sump as a recycle stream, while the reaction liquid provided to the top condenser is not, or at least not in its entirety. In one embodiment, the lactic acid concentration in the reaction liquid provided to the top of the lower condenser is such that the total acid concentration is at least 90 wt. %, in particular at least 92 wt. %, wherein the total acid content is the monomeric acid content measured after complete hydrolysis of any intermolecular ester bond with a precisely known amount of excess base and determined by back titration of remaining base with acid. In one embodiment, the lactic acid concentration in the reaction liquid provided to the top of the top condenser is such that the total acid concentration is at most 20 wt. %, in particular at most 10 wt. %, wherein the total acid content is the monomeric acid content measured after complete hydrolysis of any intermolecular ester bond with a precisely known amount of excess base and determined by back titration of remaining base with acid.

In this embodiment, the majority of the lactide present in the vapour stream is removed in the lower condenser, e.g., at least 60% of the lactide present in the feed, in particular at least 70%, more in particular at least 80%, still more in particular at least 90%. The top condenser is intended to remove lactide to a very low concentration indeed, e.g., to a total concentration in the gas stream of at most 0.07 ppm, in particular at most 0.05 ppm, more in particular at most 0.03 ppm, more in particular at most 0.02 ppm, in some cases at most 0.01 ppm.

The liquid effluent withdrawn from the condensation-reaction step comprises less than 15 wt. % lactide and less than 30 wt. % of water, and has a total acid content of at least 72 wt. %. It is preferred for the liquid effluent to be highly concentrated. Accordingly, it is preferred for the liquid effluent to comprise at most 25 wt. % of water, in particular at most 20 wt. % of water, more in particular at most 15 wt. %, still more in particular at most 10 wt. %, even more in particular at most 5 wt. %. Lower amounts, e.g. at most 4 wt. %, or at most 3 wt. %, or at most 2 wt. % are also possible. In specific applications, lower water contents are also possible, e.g., at most 1 wt. %, in some embodiments at most 0.5 wt. %.

To prevent precipitation of solid lactide, it is preferred for the lactide concentration in the liquid effluent to be at most 15 wt. %, in particular at most 10 wt. %, more in particular at most 7 wt. %, more in particular at most 5 wt. %, even more in particular at most 3 wt. %. The exact percentage that is acceptable will depend on the composition and temperature of the effluent, and can be determined by the skilled person.

The liquid effluent that is withdrawn from the condensation-reaction step preferably has a lactic acid content of at least 80 wt. %, in particular at least 85 wt. %, more in particular at least 90 wt. %, even more in particular at least 92 wt. %, more in particular at least 94 wt. %, still more in particular at least 96 wt. %, wherein the total acid content is the monomeric acid content measured after complete hydrolysis of any intermolecular ester bond with a precisely known amount of excess base and determined by back titration of remaining base with acid. Depending on the amount of water and 2+ lactic acid oligomers, the total acid content may be even higher, e.g., at least 98 wt. %, or even above 100 wt. %. As an upper limit, a value of 125 wt. %, in particular 115 wt. %, may be mentioned. As the skilled person will understand, the total acid content may be above 100 wt. % because the total acid content is carried out after hydrolysis of the oligomers.

The liquid effluent may contain further (non-lactide organic) components, e.g., further acidic components. If present, such components may generally be present in an amount of less than 5 wt. %.

In one embodiment, the liquid effluent comprises at least 20 wt. % of 2+ lactic acid oligomers (total of linear oligomers and lactide), in particular at least 30 wt. %, more in particular at least 40 wt. % in some embodiments at least 50 wt. %. This is an indication of a highly concentrated solution.

The composition of the liquid effluent is the composition upon withdrawal from the condensation-reaction step.

In addition to the liquid effluent discussed above, the condensation-reaction step also generates a steam effluent. The steam effluent contains the majority of the water present in the vapour stream comprising lactide and water that was provided to the condensation-reaction step, namely at least 60%, in particular at least 70%, more in particular at least 75 wt. %, still more in particular at least 80%, in some embodiments at least 85%, or at least 90 wt. %, more in particular at least 94%, or even at least 96%. The steam effluent and its further processing will be discussed below.

### Condensation Step Following the Condensation-Reaction Step

In the present invention steam effluent withdrawn from the condensation-reaction step is provided to a further condensation reaction in a further condensation apparatus to form a condensate comprising water. The steam effluent generally consist for at least 90 wt. % of water, calculated on the total of water, lactic acid and lactide, in particular at least 95 wt. %, in some embodiments at least 98 wt. %. This further condensation step is sometimes referred to in the present specification as the steam condensation step.

The steam condensation step is generally carried out at a temperature of between 5 and 60° C., such as between 10 and 40° C., e.g., between 12 and 30° C., or between 15 and 25° C., and at a pressure of 10 to 120 mbar, in particular 10 to 100 mbar, such as from 10 to 50 mbar, or 10 to 40 mbar.

In an example the steam condensation step is operated in a process of the invention at a temperature of 40° C. and pressure of 100 mbar. In another example, the steam condensation step is operated in a process of the invention at a temperature of 25° C. and pressure of 50 mbar. In another example, the steam condensation step is operated in a process of the invention at a temperature of 20° C. and pressure of 30 mbar. In another example, a third condensation apparatus is operated in a process of the invention at a temperature of 15° C. and pressure of 20 mbar.

The pressure in the steam condensation step is preferably produced by means of a vacuum system, for instance one or more vacuum pumps such as a liquid-ring pumps. In one embodiment, the pressure in the steam condensation step is the same as the pressure in the condensation-reaction step. This would allow using the same vacuum system for both units, which is advantageous from both an operating cost and an investment cost.

The condensate consists for the vast majority, e.g., for at least 90 wt. %, in particular at least 95 wt. %, in some embodiments at least 99 wt. % of water. It can contain traces of lactic acid, in which case it may be purified further, e.g. by feeding it to continuous contaminated sewer for treatment. Preferably, the recovered water comprises less than 10 wt. % lactic acid, in particular less than 5 wt. % lactic acid, more in particular less than 1 wt. % lactic acid, based on the total weight of the condensate formed. Optional waste gases comprising non-condensable gases and optionally small amounts of steam can be discharged via the vacuum system.

### Lactide Condensation Step

As indicated above, the feed to the condensation-reaction step is a vapour stream comprising lactide and water. This vapour stream may be derived from a lactide condensation step in which a feed comprising lactide is provided to a condensation step at reduced pressure, with the condensation step generating a lactide condensate stream and a vapour stream comprising lactide and water. In such embodiment, the vacuum in the lactide condensation step (and the upstream section) is created by the use of one or multiple steam ejectors through which the vapours out of the lactide condenser flow.

As will be evident to the skilled person, a lactide condenser is generally used when highly concentrated lactide streams are available, e.g. in the manufacture of polylactic acid (PLA), as will be discussed below. In such a case a lactide condenser is used to recover the easily condensable lactide, and the vapour stream comprising lactide and water is provided to the condensation-reaction step of the present invention. Whether a lactide stream will be provided directly to the condensation-reaction step or whether it will first be provided to a lactide condenser with a vapour stream comprising lactide and water that is recovered from the lactide condenser being provided to the condensation-reaction will depend on the concentration of lactide in the stream and the process economics of providing an additional condenser as compared to increasing the scale of the apparatus in which the condensation-reaction step is carried out. It is within the scope of the skilled person to take this decision.

The product resulting from the lactide condensation step is a condensate comprising lactide in high amounts. Preferably, the condensate recovered in the lactide condensation step comprises at least 90.0 wt %, or at least 95.0 wt %, or at least 97.5 wt %, or at least 99.0 wt% of lactide, with wt % being based on the total weight of the condensate. Hence, in certain embodiments of the present invention, the amount of the lactide in the condensate is that high such that the condensate essentially consists of lactide. The term “essentially consists of” in this context means that the condensate may comprise minimal amounts of components different from lactide that associated with the process for condensation the vapour stream, for example traces of catalyst, initiator, inhibitor, lactic acid, volatile organic acids, lactoyllactic acid (the linear dimer of lactic acid) and/or higher linear and cyclic oligomers.

The present invention allows to recover the thus separated lactide at a high yield and in high purity. The lactide may be processed as desired. Some possibilities thereof will be discussed in more detail when discussing specific aspects of the present application.

In the lactide condensation step, vapor stream comprising lactide is condensed at a temperature comprised between 150 and 96° C., such as between 100 and 120° C., or between 100 and 115° C. The temperature is to be selected such that the lactide is in the liquid phase, to prevent the presence of solid lactide interfering with process operations. The pressure in the lactide condensation step generally is at most 50 mbar, in particular at most 20 mbar, in particular at most 10 mbar, e.g., in the range of 0.5 to 5 mbar, in particular 1 to 3 mbar. In an example a lactide condensation step is carried out at a temperature of 150° C. and a pressure of 20 mbar. In another example, a lactide condensation step is carried out at a temperature of 117° C. and pressure of 5 mbar. In another example, a lactide condensation step is carried out at a temperature of 105° C. and pressure of 2 mbar. In another example, a first condensation apparatus is operated in a process of the invention at a temperature of 100° C. and pressure of 1 mbar.

In one embodiment, the lactide condensation step is carried out under such conditions that of the lactide provided to the lactide condensation step at least 70% is condensed, in particular at least 80%, more in particular at least 90%, in some embodiments at least 95%. The pressure in the lactide condensation reaction is preferably produced by means of one or more steam ejector(s), which are connected in fluid connection with the lactide condensation apparatus. In particular, in one embodiment, the water present in the vapour stream comprising lactide and water that is provided to the condensation-reaction step results at least in part, from the effluent from the lactide condenser passing though one or more steam ejectors.

The first condensation reaction can be performed in any condensation device or apparatus suitable for condensing vapor stream drawn off from the devolatilization unit.

In certain embodiments of the present invention, the first condensation apparatus has at least one outlet for discharging the condensate comprising the lactide, accumulating in the condensation apparatus. Optionally, the present process may comprise the step of collecting the condensate comprising the lactide in a collection tank for the lactide, provided downstream of the first condensation apparatus and in fluidic connection therewith. Such collection tank can serve for example for temporary storage of the lactide.

### Sources of the Vapour Stream

Vapour streams suitable as starting material for processes of the present invention can be derived from various sources.

In one embodiment, the vapour stream is derived from a process for manufacturing PLA.

Therefore, the present invention is also directed to a process for manufacturing polylactide (PLA) comprising the steps of
providing lactide to a polymerisation process which generates polylactide and a lactide-containing gas stream,
providing the lactide containing gas stream to a lactide condenser under such conditions that a liquid lactide stream and a vapour stream comprising lactide and water is generated, directly or after passing through one or more steam ejectors,
providing the vapour stream comprising lactide and water to the process according to the present invention as described herein.

In another embodiment, the vapour stream is derived from a process for manufacturing lactide. Therefore, the present invention is also directed to a process for manufacturing lactide comprising the steps of
providing lactic acid to an oligomerisation step, to generate lactic acid oligomers,
providing the lactic acid oligomers to a depolymerisation step to form a lactide mixture,
subjecting the lactide mixture to one or more purification and separation steps, to form at least one purified lactide stream,
   wherein at least one of the oligomerisation step, the depolymerisation step and the purification and separation step or steps yields a lactide-containing gas stream which, directly or after passing through one or more steam ejectors, is provided as feed to the process of the present invention.

In a further embodiment, the vapour stream is derived from a process for depolymerising polylactide (PLA), e.g., in the context of PLA recycling. Therefore, the present invention is also directed to a process for depolymerising polylactide (PLA) comprising the steps of
subjecting PLA to a depolymerisation step to provide
an effluent stream comprising one or more of lactide, lactic acid, lactic acid oligomers, and polylactic acid with a degree of polymerisation that is reduced as compared to the degree of polymerisation of the PLA that is subjected to a depolymerisation step, and
a lactide-containing gas stream,
   which process additionally generates a lactide-containing gas stream,
   wherein the lactide-containing gas stream, directly or after passing through one or more steam ejectors, is provided as feed to the process of the present invention.

Processes for manufacturing polylactide, processes for manufacturing lactide, and processes for depolymerising polylactide are in themselves known in the art and require no further elucidation here. How the condensation-reaction step and further condensation step of the process according to the invention are incorporated into these processes will depend on their pressure and water content. The process according to the invention finds particular application in feeds which are derived from a steam ejector.

### Lactide Stream Derived From the Manufacture of Polylactic Acid (PLA)

The process according to the invention finds particular application in the manufacture of polylactide, also indicated herein as polylactic acid or PLA, where lactide that is evacuated from a polylactide melt is to be recovered from a gas stream. This embodiment will thus be discussed in detail below.

Generally, two alternative methods for synthesizing polylactic acid are known. The first method is the direct polycondensation of lactic acid to polylactic acid, which leads to low molecular weight polymer only. The second method is the ring-opening polymerization of lactide. In preferred embodiments of the present process, the PLA melt to be treated is obtained via a ring-opening polymerisation of lactide. In such method, lactide monomer is polymerised in the presence of catalyst and optionally an initiator in a reactor to form a reaction mixture which comprises the resulting polylactide in a molten phase and unreacted lactide. Such resulting reaction mixture may also contain lower amounts of oligomers and residual catalyst and/or initiation. The reaction mixture comprising the PLA melt is then subjected to devolatilization, to obtain a purified PLA as molten residue and a vapor stream. This vapor stream mainly includes lactide, but may also contain remaining catalyst and/or initiator and/or a reaction product or residues thereof.

Devolatilization methods are usually based on applying low pressure and/or inert gas flow together with temperatures sufficiently high to cause separation of unconverted lactide monomer from the PLA melt by evaporation or volatilisation. In accordance with certain embodiments of the present invention, removal of unreacted lactide can be achieved by means of at least one devolatilization step conducted at elevated temperature and reduced pressure. For instance, the devolatilization reaction may be carried out at a temperature of 170° C. to 250° C., such as at a temperature of 180° C. to 240° C. or at a temperature of 190° C. to 230° C., or at a temperature of 210 to 225° C., and a pressure of 0.1 to 50 mbar, such as 0.5 to 25 mbar, or 1 to 10 mbar, or 1 to 5 mbar, or from 1 to less than 3 mbar. In particular, the devolatilization may be performed at a temperature of 170° C. to 250° C. and at a pressure of 0.1 to 50 mbar, preferably at a temperature of 180° C. to 240° C. and at a pressure of 0.5 to 25 mbar, more preferably at a temperature of 190° C. to 230° C. and at a pressure of 1 to 10 mbar, even more preferably at a temperature of 210 to 225° C. and at a pressure from 1 to less than 3 mbar.

Suitable equipment appropriate for the devolatilization of PLA is known in the art and requires no elucidation here. The devolatilization step may be carried out in one or more devolatilization units. Each devolatilizer may comprise an melt inlet for supplying a PLA melt to the devolatilizer, and vapour outlet, for removing a vapour stream from the devolatilizer.

In one embodiment of the invention, the lactide-containing vapour stream is provided to the lactide condensation step discussed above.

The vacuum (pressure) applied in the devolatilization unit is preferably produced by means of one or more steam ejector(s), which are in fluidic connection with the devolatilization unit.

A cascade of two or more steam ejectors may be used as well. In particular, the arrangement of a plurality of steam ejectors in a cascade enables the production of low pressures, which enables efficient separation of a lactide-containing vapour from the PLA melt.

In general, the unit in which the lactide condensation step discussed above is carried out is positioned between the devolatilization unit and the one or more steam ejectors, and the devolatilization unit is in fluidic connection unit in which the lactide condensation step discussed above is carried out, which in turn is in fluidic connection with the one or more steam ejector(s). The devices, i.e. devolatilization unit, lactide condensation unit, and one—or more steam ejector(s), are thereby connected successively in series so that, by means of the low pressure produced by the one or more steam ejector(s), gases or vapours can be conducted out of the devolatilization unit to the lactide condensation apparatus.

In certain embodiments of the present process, a two-stage devolatilization process can be performed in order to obtain the required degree of lactide removal and thus to obtain a polymer having the required quality. In such embodiments, the devolatilization step may be carried out in at least two devolatilization apparatuses (devolatilizers) provided in series, and vapour streams may be collected from each of said devolatilization apparatuses. The vapour streams may be combined, and provided to the lactide condensation step in combination. It is of course also possible to send them to individual lactide condensation steps.

In one embodiment, the invention provides a single set up in which at least two devolatilizers are connected to one or more lactide condensation units, which are in turn connected to a single stream ejector set up. This embodiment allows a single steam ejector set-up to regulate the pressure in a number of devolatilizers and lactide condensation units, which is efficient from both an investment and an apparatus point of view.

A devolatilization step as applied in the present process permits to separate a purified PLA melt. In certain preferred embodiments, a purified PLA melt obtained in accordance with the present invention contains less than 1.0 wt % of lactide (unreacted lactide), and preferably less than 0.5 wt % of lactide, such as between 0.01 and 0.4 wt % of lactide, or between 0.01 and 0.3 wt % of lactide, with wt % based on the total weight of the PLA melt.

The lactide content in a PLA melt that may be obtained in a process according to the invention may be determined by a precipitative method to separate the monomeric lactides from the polylactide. To that end, a polylactide sample is dissolved in a known amount of dicholoromethane (including an internal standard). The polylactide fraction of the sample is then removed by precipitation by introducing the dichloromethane solution into an excess amount of 5/95 acetone/hexane solvent mixture. After half an hour of precipitation, the polymeric fraction is removed by filtration over a 0.45 μm filter. The remaining solution is analysed using Gas Liquid Chromatography, to determine the amount of lactide monomers in the sample. The final amount of residual lactides is calculated by taking the sum of L-, D-and meso-lactide.

### Products

The process of the present invention, and its various embodiments, generate a number of process streams, which may be processed as desired.

The process generates a liquid effluent from the condensation-reaction step as discussed above. The liquid effluent has a high lactic acid concentration, and a relatively low content of non-lactic acid components. The liquid effluent can, e.g., be provided to an oligomerisation step in a process for manufacturing lactide which may in turn be provided to a PLA manufacturing process, or to a process for manufacturing lactic acid co-polymers. It can also be provided to other processes and applications where lactic acid can be used. It can also be provided to process steps where a lactic acid solution is used as washing or reaction liquid.

The process further generates a condensate comprising water. The condensate generally has a high purity and can be disposed of or re-used. If the condensate contains minor amounts of lactic acid, it can be purified before further processing.

Where the process according to the invention comprises a lactide condensation step, a lactide stream will also be produced. This lactide stream can, e.g., be provided to a PLA manufacturing process, or to a process for manufacturing lactic acid co-polymers. The lactide stream can also be provided to a lactide manufacturing process.

### Figures

The present invention is explained in more detail with reference to the accompanying figures.

The figures are for illustration purposes only; the invention is not limited thereto or thereby.

The following is noted with respect to the figures:

Embodiments of various figures can be combined unless they are mutually exclusive. The figures are flow sheets illustrating the process according to the invention. The figures do not present a reactor set up. For example, where a separation step is shown in a single step, it may be carried out on more than one reactor. Conversely, different steps may be carried out in the same unit. By the same token, the various lines are intended to show how components flow from one reaction step to the other. They do not represent real-life structures. The figures are not intended to show specific engineering features or details, including the design of the various components shown.

The figures do not always show all elements of the process according to the invention.

The figures do not show all purge streams or make-up streams that may be present in the practical performance of the process according to the invention although, as will be evident to the skilled person, purge streams and make-up streams may be necessary in practice to maintain stable operation. In addition, auxiliary equipment such as various valves, (vacuum) pumps, heating and cooling equipment, including inlets and outlets for cooling media, analytical devices, control devices and the like are not always shown in the figures, but of course such equipment can be used as necessary or desirable, and is well known the skilled person.

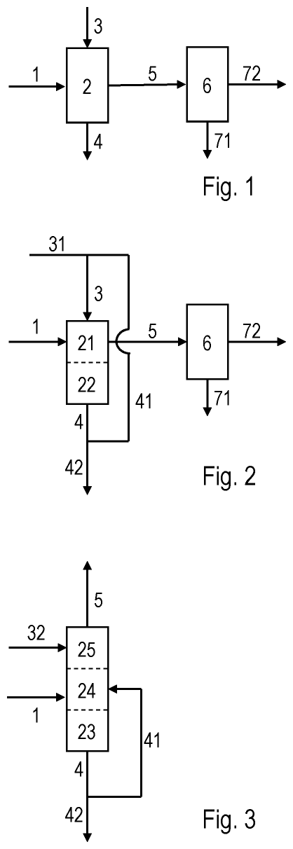

FIG. 1 illustrates a first embodiment of the present invention. In FIG. 1, a vapour stream comprising lactide and water is provided through line (1) to a condensation-reaction step in a condensation-reaction step (2). A lactic acid containing reaction liquid is provided through line (3). A liquid effluent comprising 20-80 wt. % of 2+ lactic acid oligomers (total of linear oligomers and lactide), 20-75 wt. % of lactic acid, and less than 30 wt. % of water is withdrawn from the condensation-reaction step through line (4). A steam effluent is withdrawn from the condensation-reaction step through line (5), and provided to a condensation apparatus (6) to form a condensate comprising water, which is withdrawn from the condensation apparatus through line (71). A gaseous effluent is withdrawn through line (72).

FIG. 2 illustrates a variation on FIG. 1, in which the condensation-reaction apparatus comprises two sections, namely an absorption section (21) and a reaction section (22). The feed comprising lactide and water is provided through line (1) to the absorption section (21) where it is contacted with a lactic acid containing reaction liquid provided through line (3). The reaction liquid in which lactide has been absorbed passes to reaction section (22). Liquid effluent comprising withdrawn from the condensation-reaction step through line (4). Part of the liquid effluent is withdrawn from the process through line (42). Another part of the liquid effluent is recycled though line (41) as reaction liquid to absorption section (21). In the embodiment as presented in FIG. 2, this is done by providing it to line (3), where it may be combined with stream provided through line (31), which may comprise water and/or lactic acid. The provision of stream (31) is optional.

FIG. 3 illustrates a specific embodiment of a unit suitable for performing a condensation-reaction step in the process of the invention. The unit comprises three sections, namely a sump (23), a lower condenser (24), and a top condenser (25). The vapour feed comprising lactide and water is provided through line (1) to the lower end of the lower condenser (24), and travels upwards. Reaction liquid is provided through line (41) as recycle liquid from the sump (23) to the upper end of the lower condenser (24) through a liquid distributor (not shown). Lactide is absorbed in the reaction liquid, which falls down to the sump, where the lactide is reacted. The vapour flow from which part of the lactide has been removed flows upwards to top condenser (25), where it is contacted with a lactic-acid containing reaction liquid provided through line (32) via a liquid distributor (not shown). This reaction liquid absorbs lactide present in the gas in the top condenser. The reaction liquid flows down to the lower condenser (24) where it picks up further lactide, and ultimately flows down to the sump (23). Steam effluent is withdrawn from the upper end of the top condenser though line (5) and provided to a further condenser (not shown).

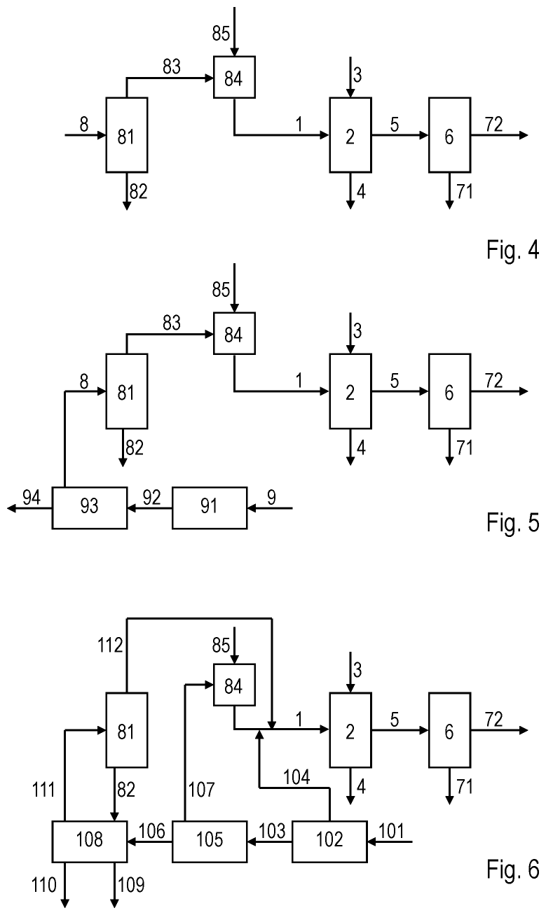

FIG. 4 illustrates a further embodiment of present invention, in which the condensation-reaction step is preceded by a lactide condensation step in which the vacuum is provided through a steam ejector. Again, it is a variation on FIG. 1.

In this figure, a lactide-containing feed is provided through line (8) to lactide condenser (81). A liquid lactide stream is withdrawn from the lactide condenser (81) through line (82). A gaseous effluent comprising a minor amount of lactide is withdrawn through line (83), and passes through a steam ejector (84) to which steam is provided through line (85). Steam ejector (84) may be a single apparatus, but it may also consist of a number of individual apparatus. Steam ejector (84) generates a low pressure in lactide condenser (81), and depending on how it is connected it may be used to create a low pressure in other apparatus also. Steam ejector (84) produces a vapour stream comprising lactide and water which is provided through line (1) to the condensation-reaction step (2).

FIG. 5 illustrates an embodiment of the present invention in which the process according to the invention is integrated into a PLA manufacturing process. It corresponds to FIG. 4, but additionally shows the following: A lactide containing feed is provided through line (9) to a polymerisation unit (91). In polymerisation unit (91) PLA is manufactured, and withdrawn through line (92) in the liquid phase. The PLA is provided to a devolatilization step (93), which results in a purified PLA stream (94) and a gaseous lactide stream (8). This gaseous lactide stream is provided to lactide condenser (81). In this embodiment, the liquid lactide stream withdrawn from the lactide condenser through line (82) may be provided, if so desired, to the lactide containing feed in line (9) or to polymerisation unit (91), either directly or after one or more intermediate steps, e.g. purification or separation steps. It can also be provided to other units.

FIG. 6 illustrates an embodiment of the present invention in which the process according to the invention is integrated into a lactide manufacturing process. In FIG. 6 a lactic acid stream is provided through line (101) to an oligomerisation step (102), where lactic acid is subjected to a condensation reaction to form lactic acid oligomers, generally with a degree of polymerisation of 2-20. The oligomerised lactic acid product is withdrawn through line (103). A vapour stream, which may comprise some lactide, is withdrawn through line (104). The oligomerised lactic acid product in line (103) is provided to depolymerisation step (105). In depolymerisation step (105), the lactic acid oligomers are depolymerised to form lactide, which is withdrawn through line (106), and provided to separation/purification step (108). A lactide-containing gas steam (107) is also formed. In separation/purification step (108) contaminants will be removed from the lactide, and the lactide will be separated to form various lactide streams of different compositions. In the schematic representation of FIG. 6, there is a first lactide stream (109) and a second lactide stream (110). The exact number of streams will depend on the exact process configuration. The separation/purification step (108) also generates lactide-containing gas streams. The schematic representation of FIG. 6 shows stream (111). The separation and purification as it occurs in the manufacture of lactide may generate a number of gas streams containing lactide, of which one or more may be provided to lactide condenser (81), to steam ejector (84), or to condensation-reaction apparatus (2). In the schematic embodiment illustrated in FIG. 6, lactide stream (104) derived from oligomerisation step (102) is provided to the condensation reaction step (2) by feeding it to line (1). Lactide-containing gas stream (107) derived from depolymerisation step (105) is provided to steam ejector (84). Lactide-containing gas stream (111) derived from separation/purification step (108) is provided to lactide condenser (81). In the embodiment provided in the figure, the condensed lactide stream (82) is recycled back to separation/purification step (108), while gaseous effluent stream (112) comprising a minor amount of lactide is provided to the condensation reaction step (2) by feeding it to line (1). The selection of the destination of the various streams will depend on their lactide content and the pressure at which they are. It is possible for not all of these steams to be present, or for the steams to be disposed of differently than in the figure.

As will be evident to the skilled person, preferred embodiments of different aspects of the present invention can be combined, unless they are mutually exclusive.

Suitable determination methods for the various parameters are within the scope of the skilled person. The compounds at issue are conventional, and determination methods for them are well known. Lactide and lactide oligomers can be determined using HPLC. Total acid of a composition is the monomeric acid content measured after complete hydrolysis of any intermolecular ester bond with a precisely known amount of excess base and determined by back titration of remaining base with acid.

The amount of lactide absorbed in reaction liquid can be calculated from the amount of lactide provided to the condensation-reaction step and the amount of lactide present in the steam effluent withdrawn from the condensation-reaction step. The amount of water in the liquid effluent can be calculated by subtracting the amount of lactide and optional other compounds from the total weight of the fraction. Another suitable method to determine the amount of water would be to use a Karl Fischer titration.

The present invention is illustrated by the following examples, without being limited thereto or thereby.

### Example 1

In a continuous process, a vapour feed comprising lactide and water is provided to a condensation-reaction step, wherein the feed is contacted with a lactic acid-containing reaction liquid. The contacting takes place at a pressure of 30 mbar and a temperature of 60° C. The reaction liquid is recycled over the unit until the liquid reaches the following composition: less than 5 wt. % water, and less than 5 wt. % lactide, and a total acid content of at least 90 wt. %. The product contains less than 5 wt. % non-lactide components. Once this composition is reached, part of the liquid effluent is withdrawn from the process, while another part of the liquid effluent continues to be recycled as reaction liquid.

The process is operated under such conditions that more than 95% of the lactide in the feed is captured in the reaction liquid, while less than 20 wt. % of the water in the feed is captured in the reaction liquid.

The condensation-reaction step also yields a steam effluent, which is provided to a steam condenser, operated at a temperature of 30 mbar and 20° C. In this condenser, the steam is condensed to form a water fraction with a lactic acid content below 5 wt. %.

The feed is derived from a lactide condenser operated at a temperature of 105° C. and a pressure of 2 mbar. The pressure is generated by a steam ejector, resulting in the presence of water in the feed to the condensation-reaction step.

1. A process for removing lactide from a vapor stream comprising lactide and water, comprising the steps of
subjecting a vapor stream comprising lactide and water to a condensation-reaction step in a condensation-reaction apparatus, wherein the vapor stream comprising lactide and water is contacted with a reaction liquid comprising lactic acid, the condensation reaction step being carried out under such conditions that at least 80% of the lactide present in the vapor stream provided to the condensation-reaction step is absorbed in the reaction liquid and at most 40% of the water present in the vapor stream provided to the condensation-reaction step is absorbed in the reaction liquid, wherein the residence time in the condensation step is at least 2 hours, the residence time being defined as the total mass of reaction liquid divided by the mass of the product condensing from the vapor stream containing lactide and water per hour,
withdrawing a liquid effluent from the condensation-reaction step, which liquid effluent comprises less than 15 wt. % lactide and less than 30 wt. % of water, the effluent having a total acid content of at least 72 wt. %, and
withdrawing a steam effluent from the condensation-reaction step, and providing the steam effluent to a further condensation reaction in a further condensation apparatus to form a condensate comprising water.

2. The process according to claim 1, wherein at least 90% of the lactide present in the vapor stream provided to the condensation-reaction step is absorbed in the reaction liquid.

3. The process according to claim 1, wherein at most 25% of the water present in the vapor vapour stream provided to the condensation-reaction step is absorbed in the reaction liquid.

4. The process according to claim 1, wherein the residence time in the condensation-reaction step is at least 5 hours, and at most 1000 hours.

5. The process according to claim 1, wherein the liquid effluent comprises at most 25 wt. % of water.

6. The process according to claim 1, wherein the lactide concentration in the liquid effluent is at most 15 wt. %.

7. The process according to claim 1, wherein the total acid content of the liquid effluent is at least 90 wt. %, wherein the total acid content is the monomeric acid content measured after complete hydrolysis of any intermolecular ester bond with a precisely known amount of excess base and determined by back titration of remaining base with acid.

8. The process according to claim 1, wherein at least 50% of the lactide provided to the condensation-reaction step is converted into lactic acid or lactic acid oligomers as calculated from the amount of lactide provided to the condensation-reaction step and the lactide content of the lactic acid-containing liquid withdrawn from the condensation-reaction step.

9. The process according to claim 1, wherein the condensation-reaction step is carried out in a single unit, optionally having different sections.

10. The process according to claim 1, wherein the condensation-reaction step is carried out in a number of steps in a number of units, wherein the units are the same of different.

11. The process according to claim 1, wherein the condensation-reaction step is carried out at a temperature in the range of at least 20° C., and at most 150° C., and a pressure of at most 100 mbar and at least 20 mbar

12. The process according to claim 1, wherein the vapor stream comprising lactide and water is derived from a lactide condensation process wherein a lactide-containing gas feed is provided to a lactide condenser under such conditions that a liquid lactide stream and a vapor stream comprising lactide and water is generated, wherein the water in the vapor stream comprising lactide and water is preferably derived from a lactide-containing gas stream passing through one or more steam ejectors.

13. A process for manufacturing polylactide comprising the steps of
providing lactide to a polymerisation process which generates polylactide and a lactide-containing gas stream, and
providing the lactide-containing gas stream to a lactide condenser under such conditions that a liquid lactide stream and a vapor stream comprising lactide and water is generated, directly or after passing through one or more steam ejectors, and
providing the vapor stream comprising lactide and water to the process according to claim 1.

14. A process for manufacturing lactide comprising the steps of
providing lactic acid to an oligomerisation step to generate lactic acid oligomers,
providing the lactic acid oligomers to a depolymerisation step to form a lactide mixture, and
subjecting the lactide mixture to one or more purification and separation steps, to form at least one purified lactide stream,
wherein at least one of the oligomerisation step, the depolymerisation step and the purification and separation step or steps yields a lactide-containing gas stream which, directly or after passing through one or more steam ejectors, is provided as feed to the process of claim 1.

15. A process for depolymerising polylactide (PLA) comprising the steps of
subjecting PLA to a depolymerisation step to provide an effluent stream comprising one or more of lactide, lactic acid, lactic acid oligomers, and polylactic acid with a degree of polymerisation that is reduced as compared to the degree of polymerisation of the PLA that is subjected to a depolymerisation step, and a lactide-containing gas stream,
wherein the lactide-containing gas stream, directly or after passing through one or more steam ejectors, is provided as feed to the process of claim 1.
